(19) United States
(12) Patent Application Publication
(10) Pub. No.: US 2009/0306604 A1
(43) Pub. Date: Dec. 10, 2009

(54) Hybrid cannula/electrode medical device having protected wire passage

(76) Inventors: George P. Darmos, Willowdale (CA); Iiya Gavrilov, Etobicoke (CA); Peter G. Darmos, Willowdale (CA)

Correspondence Address: BERESKIN AND PARR LLP/S.E.N.C.R.L., s.r.l., 40 KING STREET WEST, BOX 401, TORONTO, ON, M5H 3Y2, CA

(21) Appl. No.: 12/155,586

(22) Filed: Jun. 6, 2008

(51) Int. Cl. A61M 5/32; A61B 18/18

(52) U.S. Cl. 604/264; 606/41

(57) ABSTRACT

A hybrid cannula having first and second passages extending along its length, the first passage to transport liquid to be injected into a patient and the second passage to hold an electric wire. The cannula is formed by at least two concentric metal tubes. The liquid passage extends through the inner tube, while the wire passage extends through either the outer surface of the inner tube or through a middle tube, such that the outer periphery of the wire passage is always covered by the metal wall of the outer tube. Thus, the wire can be inserted without difficulty through the second passage and does not contact the insulation layer. This allows formation of the insulation layer with a smooth outer surface.

### FIELD

This invention relates to an instrument to be used in medical applications, in which energy (usually radio frequency energy) is to be emitted in the vicinity of tissue in a patient, and to a method of making the instrument.

### BACKGROUND

A common method of treatment of patients involves inserting an electrode or electrodes into an insulated cannula that has a sharp, beveled, exposed bare tip so that the cannula can penetrate tissue. The cannula is inserted into the body and is guided toward tissue to be treated. The same cannula that accepts the electrode can also be used to deliver an anesthetic liquid, or a diagnostic localization agent, to the target structure or tissue in the body. Our U.S. Pat. No. 7,318,822 shows a hybrid cannula/electrode structure which can be used to deliver both a necessary liquid, and RF energy, to the tissue at the same time without using two separate devices. This is accomplished by providing two separate passages in the device, one passage for an electrical wire or wires, and the other passage for the liquid to be injected.

However, it will be realized that the outer surface of the hybrid cannula must be very smooth, since it must penetrate a patient's tissue and is usually threaded through a blood vessel such as an artery. Bumps or discontinuities in the outer surface would be highly undesirable. A problem which has arisen in constructing a hybrid cannula such as that shown in our prior U.S. Pat. No. 7,318,822 is that the wire which extends along the cannula, usually in a crease or groove in its outer surface, is very difficult to insert in such groove or crease. If the wire does not lie smoothly and uniformly within the groove or crease, then after the insulation (usually a heat shrink film) is applied to the outer surface of the cannula and is heated and thereby shrunk, the outer surface of the cannula in the vicinity of the wire will not be smooth. This is not desirable.

### SUMMARY

Accordingly, it is an object of this invention to provide a hybrid cannula of a design which can more easily be constructed without bumps or other discontinuities in its outer surface.

In one aspect the invention provides a cannula comprising:

a) at least two hollow tubes, each having a proximal and a distal end, said tubes being nested one within the other and being concentric, one of said tubes being an outer tube of electrically conductive metal and another of said tubes being an inner tube,

b) said inner tube having a first interior passage extending longitudinally therethrough and adapted to carry a liquid through said cannula,

c) at least two of said tubes cooperating to form a second interior passage extending longitudinally through said cannula, said second passage having a radially outer side, said radially outer side being entirely covered by a said tube,

d) at least one electrical wire extending longitudinally through said second passage,

e) an insulation layer covering the outer surface of said outer tube and not contacting said wire or wires, said insulation layer having a smooth outer surface.

Further objects and advantages of the invention will appear from the following description, taken together with the accompanying drawings.

### BRIEF DESCRIPTION OF THE DRAWINGS

In the accompanying drawings:

FIG. 1 is a diagrammatic side view of prior art apparatus with which the invention may be used;

FIG. 2 is a diagrammatic view showing electrical connections of a typical prior art cannula to a patient and to an energy source;

FIG. 3 is a cross-sectional view of a cannula according to the invention;

FIG. 4 is a longitudinal sectional view of the cannula of FIG. 3;

FIG. 5 is a cross-sectional view of another embodiment of a cannula according to the invention;

FIG. 6 is a longitudinal sectional view of the cannula of FIG. 5; and

FIG. 7 is a longitudinal sectional view of a modified cannula according to the invention.

### DETAILED DESCRIPTION

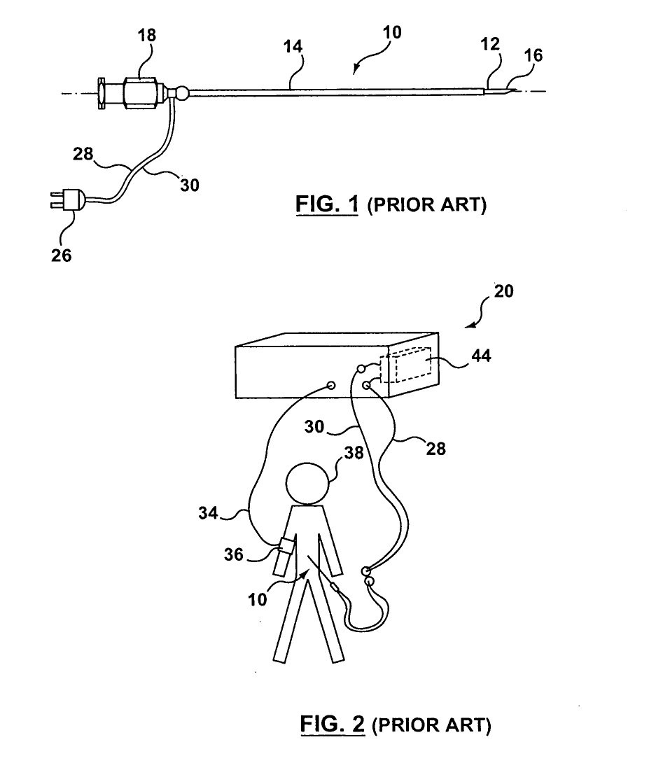

Reference is first made to FIGS. 1 and 2, which show generally apparatus with which the invention may be used. These drawings are the same as FIGS. 1 and 2 in our prior U.S. Pat. No. 7,318,822, and the drawings and description of that patent are incorporated by reference into this document for better understanding of the background.

FIG. 1 shows a cannula 10 formed from medical grade tubing 12 such as medical grade stainless steel covered almost entirely by medical grade insulation 14. The insulation is typically a conventional heat shrink film which is readily available on the market. The insulation does not cover and thus exposes a polished bare tip 16 at the distal or free end of the cannula. There is also usually a hub 18 (normally of plastic) mounted to the inner or proximal end of the cannula 10.

The cannula 10 can receive energy from an energy source such as an RF lesion generator 20, and can deliver that energy to the bare tip 16. As diagrammatically shown, an electrical connector 26 is connected by two wires 28, 30 to the cannula 10. One of the wires 28 is electrically connected (e.g. by silver solder or spot-welding) to the metal tube 12 adjacent the proximal end of the cannula. The wire 28 conducts electrical energy from the energy source 20 into the cannula 10. The other half of the circuit for the RF generator is constituted by a further conductor 34 from the RF generator 20 to a dispersive pad 36 on the patient 38, as shown in FIG. 2.

The second wire 30 from connector 26 serves as part of the temperature sensor circuit. The second wire 30 extends along the length of the cannula in a manner to be described, and as shown in the drawings, terminates adjacent the bare tip 16 at a hot junction. The hot junction consists of the wire 30, which can be copper or constantin, that is electrically connected (by solder or spot-welding) to the metal tube, so that the other half of the circuit for the thermocouple is formed by the conductive metal tube and the wire 30. If desired, a different heat sensor can be used and wire 30 can be provided as two wires, as will be described.

Thus, the cannula 10 can receive energy from the energy source 20 and can deliver that energy to the bare tip 16. The device can also relay temperature information from the bare tip 16 to the energy source controller 44, so that the amount of RF energy applied can be controlled in a well-known manner.

The hub 18 can be affixed to the cannula directly, coaxially with the tube 12 as shown in FIG. 1. An appropriate liquid, such as an anesthetic, can be injected through the hub and through the tube 12 to the tip 16. Other liquids, such as appropriate diagnostic agents, can be injected.

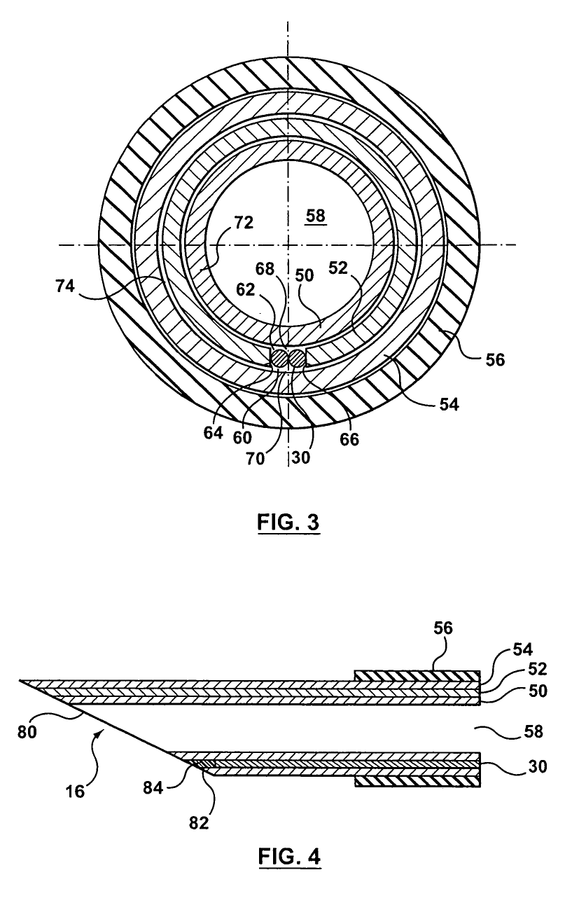

As previously stated, it is important that any wires which are present within the cannula structure be located away from the insulated surface of the cannula, so that they will not cause surface irregularities. Reference is therefore next made to FIGS. 3, 4 which show an arrangement for accomplishing this objective.

As shown in FIGS. 3, 4, the cannula 10 includes inner, middle and outer concentric metal tubes 50, 52 and 54. The inner tube 50 is nested within the middle tube 52, and the middle tube 52 is nested within the outer tube 54. The tubes are preferably sized to be a snug but not too tight fit within each other. The outer tube 54 is covered by heat shrink insulation film 56.

As shown, the inner tube 50 has an interior passage 58 through its interior, and through which the desired liquid to be injected is conducted. The middle tube 52 includes a channel or gap 60 cut or formed throughout its length, defining a second passage 62. The longitudinal radial edges of the passage 62 are defined by the two longitudinal cut edges 64, 66 of the middle tube 52. The circumferential sides 68, 70 of the passage 62 are defined (and closed) by the outer surface 72 of the inner tube 50 and by the inner surface 74 of the outer tube 54.

The second passage 62 provides the necessary opening in which one or more wires 30 can be placed to extend between the bare tip 16 of the cannula and hub 18 at its proximal end.

During assembly of the cannula shown in FIGS. 3, 4, the three metal tubes 50, 52 and 54 are nested and cut to the desired length with the distal end being cut at an angle to produce the sharpened end shown in the drawings. The three tubes are normally secured together by spot-welding (not shown) at the proximal end of the cannula.

The outer insulation layer 56 can be applied before or after the wire or wires 30 are inserted. In either case, the insulation layer 56 (formed by a plastic film as indicated) is wrapped around the cannula outer tube 54 and the entire device is then placed in a heated oven for a time sufficient to shrink the insulation layer tightly onto the outer tube 54. This method of applying insulation to a cannula is well-known in the art.

The wire or wires 30 are placed in the second passage 62 by being pushed through that passage after the three tubes 50, 52, 54 have been assembled. Normally the wires 30 will have sufficient stiffness for this purpose. If for any reason the wire or wires 30 are not sufficiently stiff to be pushed through the passage 62, then a stiff thin rod (not shown) can be attached to the ends of the wires 30 and can be used to draw them through the passage 62.

The wires 30 are moved through the passage 62 until they project a small distance (e.g., two or three millimeters) past the end of the tip 16. The two wires 30 are then spot-welded or soldered together. Once the wires have been secured together, they are pulled back so that they are recessed by about one-half to one millimeter from the end 80 of the tip 16. The recess is indicated at 82 in FIG. 4. The wires 30 are then bonded in position by a suitable insulating adhesive 84 placed in the recess 82.

While the wires 30 project forwardly of the tip end surface 80, the insulation on them is stripped off so that the wires can be soldered or spot-welded together.

A major advantage of the cannula shown in FIGS. 3, 4 is that it is constructed of modular components (the tubes 50, 52 and 54) and can be easily assembled. Most importantly, placing the wire or wires 30 within the second passage 62 is a simple and non-time-consuming operation, and since the wire or wires 30 are surrounded by a metal tubing and cannot touch the insulation layer 56, they cannot interfere with the process of creating a smooth outer surface for the insulation layer 56. The second passage 62 can be made as large as may be necessary (within the overall size limits of the cannula) to accommodate the wire or wires 30.

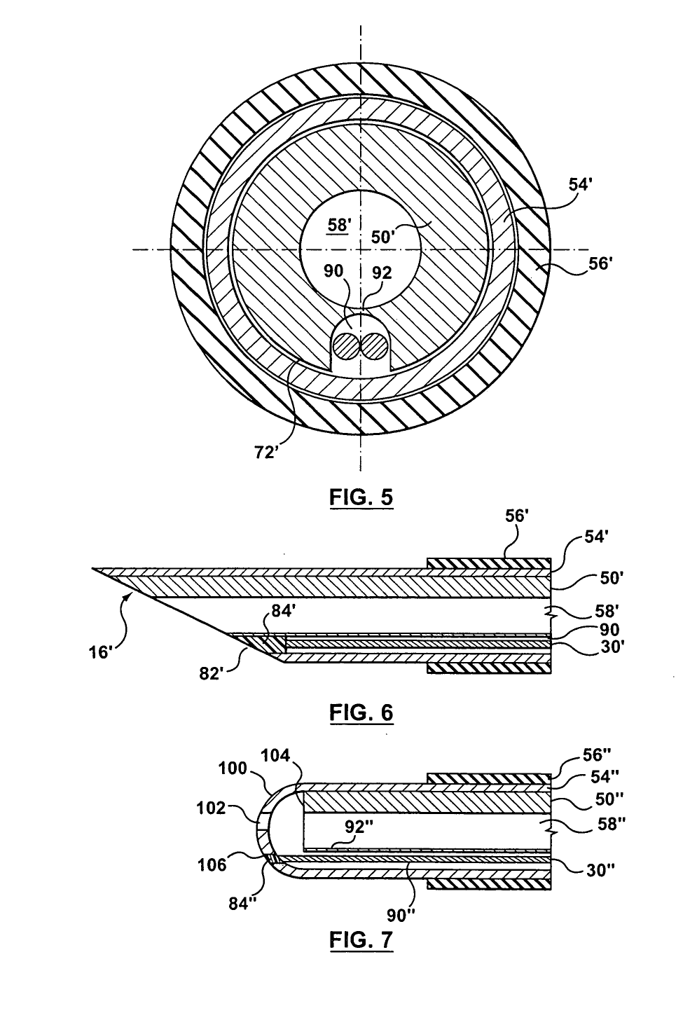

Reference is next made to FIGS. 5, 6, which show a modification of the cannula shown in FIGS. 3, 4, and in which primed reference numerals indicate parts corresponding to those of the previously described version.

In the FIGS. 5, 6 version, only two tubes are used, namely, an inner tube 50′ and an outer tube 54′. These two tubes are again concentric, with the inner tube 50′ nested within the outer tube 54′. The inner tube 50′ is thicker than the inner tube 50 of FIGS. 3, 4 and effectively comprises the middle tube 52 and the inner tube 50 of the previous embodiment, formed together as a single thick walled inner tube. The inner tube 50′ has a relatively deep groove 90 or wire passage machined or otherwise formed in its outer surface 72′, but not extending fully through the thickness of the inner tube wall. This leaves a layer 92 of metal separating the inner passage 58′ of the inner tube 50′ from the wire passage 90.

Assembly of the embodiment shown in FIGS. 5, 6 is essentially the same as described for the previous version. Firstly, the inner and outer tubes are nested together, and are cut to the desired length while forming a sharpened tip 16′ as before. The tubes 50′, 54′ may then be spot-welded at their proximal ends (not shown). The insulation layer 56′ may then be applied, or it can be applied after insertion of the wire or wires 30′.

As before, the wire or wires 30′ are pushed or drawn through the second or wire passage 90 until they protrude a short distance beyond the tip 16′. The protruding portions of the wires 30′ are then stripped of their insulation, spot-welded or soldered together, and then are pulled back until they are recessed slightly from the end of the uninsulated tip 16′. The recess, indicated at 82′ in FIG. 6, may then be filled with adhesive 84′, to prevent the wires 30′ from inadvertently being withdrawn from the passage 62′, and to protect the ends of the wires 30′ and prevent undesired material from filling the recess 82′.

Again, by using multiple tubes to form a metal enclosure for the wires 30′, which enclosure completely surrounds the wires and separates them from the insulation layer 56′, it is possible to achieve a smooth outer surface on the insulation layer 56′, without discontinuities which could cause difficulty.

While the embodiments shown have a sharp beveled tip, this can be changed in cases where the device does not need to penetrate tissue. In that case, a version such as that shown by way of example in FIG. 7 can be used. In FIG. 7, double primed reference numerals indicate parts corresponding to those of the previous embodiments.

In the FIG. 7 version, the outer tube 54″ has a rounded bare tip 100. Such round tips are commonly used on cannula and are formed in conventional manner, as is well known in the art. The rounded tip 100 includes an opening 102 through which liquid travelling through the inner passage 58″ may leave the cannula.

The inner tube 50″ nests within the outer tube 54″ as before, but the inner tube 50″ ends at a straight end 104. As before, the wall 92″ separates the inner passage 58″ from the wire passage 90″.

The wire or wires 30″ are passed through the wire passage 90″, past the open distal end of the wire passage 90″ and into a second opening 106 in the rounded tip 100. As before, the wires 30″ are pulled beyond the tip 100 sufficiently to spot weld or solder them together, and are then pulled back until they are slightly recessed within the opening 106. Then, as before, the remainder of the opening 106 is filled with adhesive 84″.

The insulation layer 56″ is heat shrunk over the cannula as before, leaving the tip bare as shown in FIG. 7.

It will be seen that in the FIG. 7 version, liquid injected into the center opening 58″ can enter the wire passage 90″. This is not a concern since the passages, and also the clearance between the inner and outer tubes, are all sealed at the hub 18 so that no fluid can escape. In addition, the hybrid cannula described is disposable, so the presence of any remnants of liquid within the cannula after use is not a concern.

However, if it is desired to reduce or eliminate the entry of liquid into the wire passage 90″, then the wire passage 90″ can be plugged at its distal end (e.g. by adhesive) before the cannula is assembled, and the wire or wires 30 can be brought out through an opening (not shown) in the side wall of the outer tube 54″. Such opening on the side wall of the outer tube 54″ at a location on the bare tip 100 (where there is no insulation 56″) is a common location for a temperature sensor wire or wires. In assembly, the wire or wires 30 are drawn through the side opening, after which the wire insulation is removed from the wire tips; the wires are soldered or spot welded together and are then drawn back into the recess provided by the side opening, and are then covered by more adhesive located within the recess.

Finally, while all of the tubes have been referred to as being of metal (specifically, electrically conductive metal), it will be realized that only one tube (normally the outer tube 54) needs to be of metal and such tube can be used to carry RF current which is applied to the patient. The remaining tubes do not need to carry current, since the electrical current for the temperature sensor can be conducted by the wires 30. The non-conducting tube or tubes can be of a suitable plastic, e.g. they can be made from the plastic known as “Teflon” (trademark).

While preferred embodiments of the invention have been described, it will be realized that various changes can be made, and these are all intended to be included within the scope of the invention.

1. A cannula comprising:
a) at least two hollow tubes, each having a proximal and a distal end, said tubes being nested one within the other and being concentric, one of said tubes being an outer tube of electrically conductive metal and another of said tubes being an inner tube,
b) said inner tube having a first interior passage extending longitudinally therethrough and adapted to carry a liquid through said cannula,
c) at least two of said tubes cooperating to form a second interior passage extending longitudinally through said cannula, said second passage having a radially outer side, said radially outer side being entirely covered by a said tube,
d) at least one electrical wire extending longitudinally through said second passage,
e) an insulation layer covering the outer surface of said outer tube and not contacting said wire or wires, said insulation layer having a smooth outer surface.

2. A cannula according to claim 1 wherein said inner tube has an outer surface and said second passage comprises a groove in said outer surface of said inner tube, said groove being covered by said outer tube, said groove not communicating with said first passage.

3. A cannula according to claim 1 and having three said tubes, namely an inner tube having said first passage and having an outer surface, a middle tube having a gap extending along its length and positioned over said inner tube, and an outer tube positioned over said middle tube and having an inner surface, so that said second passage is defined by said gap, the outer surface of said inner tube, and the inner surface of said outer tube, said wire extending through said second passage.

4. A cannula according to claim 1 and having a sharpened tip at said distal end, said wire extending through said second passage to a position recessed between said tubes from said sharpened tip.

5. A method of assembling a hybrid cannula comprising: selecting at least two hollow tubes, nesting said tubes concentrically together so that one tube is an outer tube and has a first inner and first outer surface and another tube is an inner tube and has a second outer and a second inner surface, said outer tube being of electrically conductive metal, cutting said tubes to a required length and so that said tubes have a distal end, shrinking an insulation layer onto the outer surface of the outer tube, said inner tube having an inner passage for transport therethrough of a liquid, providing a second passage extending longitudinally through said cannula at a location between the inner surface of the inner tube and the outer surface of the outer tube such that said second passage is separated by said tubes from each of said inner passage and said insulation layer, extending at least one wire through said second passage, said wire thereby being separated from said insulation layer by said outer tube, thereby to assist in creating a smooth outer surface of said insulation layer.

6. A method according to claim 6 wherein there are two said tubes, and wherein said second passage is provided by forming a groove or channel in the outer surface of said inner tube, said groove being separated from said first passage by a wall of said inner tube and being separated from the outer surface of said outer tube by a wall of said outer tube.

7. A method according to claim 6 wherein there are three said tubes, namely said inner tube having said first passage extending therethrough, a middle tube having a longitudinal gap extending along its length, and said outer tube, positioning said middle tube between said inner and outer tubes, said gap together with the outer surface of said inner tube and the inner surface of said outer tube forming said second passage, and then passing at least one wire through said second passage, said wire being separated from said inner passage by said inner tube, said wire being separated from the outer surface of said outer tube by said outer tube, so that said wire does not contact said insulation layer, thereby enabling provision of a smooth outer surface for said insulation layer.
